(19) United States
(12) Patent Application Publication
(10) Pub. No.: US 2023/0200422 A1
(43) Pub. Date: Jun. 29, 2023

(54) LACTOCOCCUS LACTIS FOR USE IN PREVENTING OR TREATING MINERAL DEFICIENCY

(71) Applicant: Compagnie Gervais Danone, Paris (FR)

(72) Inventors: Audrey BONIFACE-GUIRAUD, Les Ulis (FR); Jean-Michel FAURIE, Jouy-en-Josas (FR); Raphaëlle BOURDET-SICARD, Palaiseau (FR)

(21) Appl. No.: 17/927,303

(22) PCT Filed: May 25, 2020
(86) PCT No.: PCT/IB2020/000509

§ 371 Date: Nov. 22, 2022

(51) Int. Cl. A23L 33/135; C12N 1/20; A61K 35/744; A23L 5/20

(52) U.S. Cl. CPC A23L 33/135; C12N 1/205; A61K 35/744; A23L 5/28; C12R 2001/46

(57) ABSTRACT

The present invention relates to the use of Lactococcus lactis suitable for preventing or treating mineral deficiency or insufficiency by increasing the bioavailability of phytate bound minerals. The present invention provides novel strains, compositions comprising said strains and methods for the preparation of such compositions.

### FIELD OF THE INVENTION

The present invention relates to the use of Lactococcus lactis suitable for preventing or treating mineral deficiency or insufficiency by increasing the bioavailability of phytate bound minerals. The present invention provides novel strains, compositions comprising said strains and methods for the preparation of such compositions.

### TECHNICAL BACKGROUND

Micronutrient malnutrition is a term used to refer to diseases caused by a dietary deficiency of vitamins or minerals. According to the World Health Organization the most common of such diseases is anemia whose primary cause is iron deficiency. The World Health Organization estimates that anemia affects 1.62 billion people worldwide and is particularly prevalent in children and pregnant women. Strategies for preventing micronutrient deficiency include interventions such as dietary improvement or modification and fortification of staple food and specific products.

Phytic acid is an anti-nutritive agent present in key staple crops that inhibits the bioavailability of minerals. Phytate is the principle storage form of phosphorus in grains, oil seeds, nuts and legumes. It is considered an anti-nutritive agent as it has a high affinity for divalent minerals (including calcium, iron, zinc, copper, magnesium and manganese amongst others), thus reducing bioavailability. In developing countries unrefined cereals and legumes are the staples of a predominantly plant-based diet and the issue of bioavailability contributes to micronutrient-malnutrition. The reduction of anti-nutritive factors such as phytates has been identified as a means to reduce the incidence of mineral-malnutrition diseases.

The use of phytases to cleave phytate chelated minerals has been proposed as a means to increase mineral bioavailability, and proposed methods include the use of phytase producing lactic acid bacteria. However such methods are based on the direct fermentation of phytate containing foods as a means to improve mineral bioavailability (WO2014016398), and consumer acceptability of fermented vegetal matters remains challenging due to their particular organoleptic characteristics.

### SUMMARY OF THE INVENTION

The present invention follows from the unexpected finding that Lactococcus lactis strains degrade phytates and can be used for increasing the bioavailability of phytate complexed minerals. Even more surprising, the phytate degrading activity of Lactococcus lactis strains is present when the strains are consumed i.e. have an in vivo effect. This potentially allows the reduction of phytate in cereal foodstuffs even when the strains are consumed separately, and also does not require that the foodstuffs are fermented by means of the Lactococcus lactis strains. This allows the Lactococcus lactis to be consumed in the form of a supplement or food product such as yogurt rather than e.g. a fermented grain product.

Thus the invention provides a means of increasing mineral bioavailability, and is particularly suited to individuals following a plant-based or flexitarian diet where bioavailability of key minerals such as iron may be limited due to phytate-rich diets. Additionally, the strain of the invention is exceptionally useful in the preparation of fermented milk products due to its milk acidification properties. This enables the preparation of fermented milk products that may be more familiar and organoleptically acceptable to consumers than fermented plant-based food products. Surprisingly the phytate degrading activity may be effective in-vitro thus obviating the need for direct fermentation of the phytate rich food products to improve mineral bioavailability.

Accordingly, the present invention provides Lactococcus lactis used for preventing or treating mineral deficiency or insufficiency by increasing the bioavailability of phytate bound minerals, as well as novel strains of Lactococcus lactis suitable for these applications, such as the Lactococcus lactis strain deposited at the CNCM under reference number CNCM I-5450. The present invention also provides compositions comprising said strains and methods for the preparation thereof.

### DETAILED DESCRIPTION OF THE INVENTION

As used herein the term “stable composition” shall be taken to mean a composition that does not present sedimentation and/or serum separation.

As used herein the term “x % (w/w)” is equivalent to “x g per 100 g”.

As used herein the terms “dairy composition”, “milk-based composition” or “dairy product” shall be taken to mean a product or composition comprising essentially of or consisting of milk or milk components and optionally further ingredients.

As used herein the term “fermented dairy” shall be taken to mean a product or composition that is the product of the acidifying fermentation of a milk-based composition by a starter culture of fermenting microorganisms, in particular bacteria, preferably lactic acid bacteria. As used herein the term “fermented milk” shall be taken to mean a product or composition derived from milk by the acidifying action of at least one lactic acid bacterium. Accordingly, as used herein a fermented dairy product can thus be a fermented milk, such as a yoghurt (e.g. a set, stirred or drink yogurt), or a fresh cheese such as a white cheese or a “petit-Suisse”. It can be also be a strained fermented milk such as a strained yoghurt (e.g. a concentrated or Greek-style yoghurt).

The terms “fermented milk” and “yogurt” (or “yoghurt”) are given their usual meanings in the field of the dairy industry, that is, products suitable for human consumption and originating from acidifying lactic fermentation of a milk substrate. These products can contain secondary ingredients such as fruits, vegetables, sugar, etc.

The expression “fermented milk” may be used to refer to fermented milks other than yogurts e.g. “Kefir”, “Kumtss”, “Lassi”, “Dahi”, “Laban”, “Filmjolk”, “Villi”, “Acidophilus milk”.

The term “yogurt” or “yoghurt” as used herein shall be taken to mean fermented milk obtained by the acidifying lactic fermentation of specific thermophilic lactic acid bacteria such as Lactobacillus delbrueckii subsp. bulgaricus and Lactococcus lactis (also referred to as Streptococcus salivarius subsp. thermophilus), which must be in the living state in the finished product at a minimum CFU. In certain countries, regulations allow the addition of further lactic acid bacteria to yoghurt such as but not limited to strains of Bifidobacterium and/or Lactobacillus acidophilus and/or Lactobacillus casei. These additional lactic acid bacteria strains are intended to impart various properties to the finished product, such as that of providing organoleptic qualities, favoring equilibrium of intestinal flora or modulating the immune system.

As used herein, the term “strained composition” shall be taken to mean a fermented composition which has been subjected to a post-fermentation separation process.

As used herein the term “spoonable” shall be taken to mean a solid or semi-solid that may be consumed by means of a spoon or other utensil.

As used herein the term “fermentation” shall be taken to mean the metabolism of a substance by microorganisms, e.g. bacteria, yeasts, or other microorganisms.

As used herein the term “cfu” or “CFU” shall be taken to be an abbreviation of the term “colony forming unit”.

As used herein the term “CNCM I-” followed by a 4 digit number shall be taken to refer to a strain deposited at the Collection Nationale de Cultures de Microorganismes (CNCM) 25 rue du Docteur Roux, Paris, France under the Budapest Treaty with an accession number corresponding to said 4 digit number, e.g. CNCM I-5450.

As used herein reference to a bacterial strain or species shall be taken to include functionally equivalent bacteria derived therefrom such as but not limited to mutants, variants or genetically transformed bacteria. These mutants or genetically transformed strains can be strains wherein one or more endogenous gene(s) of the parent strain has (have) been mutated, for instance to modify some of their metabolic properties (e.g., their ability to ferment sugars, their resistance to acidity, their survival to transport in the gastrointestinal tract, their post-acidification properties or their metabolite production). They can also be strains resulting from the genetic transformation of the parent strain to add one or more gene(s) of interest, for instance in order to give to said genetically transformed strains additional physiological features, or to allow them to express proteins of therapeutic or prophylactic interest that one wishes to administer through said strains. These mutants or genetically transformed strains can be obtained from the parent strain by means of conventional techniques for random or site-directed mutagenesis and genetic transformation of bacteria, or by means of the technique known as “genome shuffling”. In the present text, strains, mutants and variants derived from a parent species or strain will be considered as being encompassed by reference to said parent species or strain, e.g. the phrases “Lactococcus lactis” and “CNCM I-5450” shall be taken to include strains, mutants and variants derived therefrom. Accordingly, as used herein reference to a bacterial strain specified by an accession or deposit number shall be taken to encompass variants thereof having at least 95% identity (see: Stackebrandt & Goebel, 1994, Int. J. Syst. Bacteriol. 44:846-849). In a particularly preferred embodiment, said variant has at least 97% identity with the 16S rRNA sequence of said specified strain, more preferably at least 98% identity, more preferably at least 99% or more identity.

As used herein the term “substantially pure” when used in reference to a bacterial strain refers to the percent of said bacterial strain relative to the total micro-organism content. Substantially pure can be at least about 99.99%, at least about 99.90%, at least about 99.50%, at least about 99.00%, at least about 95.00%, at least about 90.00%, at least about 85.00%, or at least about 75.00%.

As used herein, a “lactic acid bacterium” is a Gram-positive, acid-tolerant, generally non-sporulating and non-respiring, either rod- or cocci-shaped bacterium that is able to ferment sugars into lactic acid.

As used herein the term “malnutrition” refers to deficiencies, excesses or imbalances in an individual's intake of energy and/or nutrients.

As used herein the term ‘undernutrition’ refers to malnutrition caused by a lack of intake of energy and/or nutrients which includes stunting (low height for age), wasting (low weight for height), underweight (low weight for age).

As used herein the term “micronutrient-malnutrition”, includes micronutrient deficiencies or insufficiencies (a lack of important vitamins and minerals) or micronutrient excess.

As intended herein, a “Phytate containing food” relates to a food or foodstuff which comprises phytate or phytic acid.

As used herein the term “plant-based diet” shall be taken to refer to a subject or individual whose weekly calorific intake is entirely or at least 70% or more from plant-based sources, more preferably at least 80%, 90% or 95%. Accordingly, the term shall be taken to encompass vegan (fully plant-based), vegetarian (plant-based and may include eggs, honey and/or dairy), semi-vegetarian and/or flexitarian (increased plant-based intake) diets.

As Used Herein the Term “Plant-Based” Shall be Taken to Mean a Composition or Product which does not Comprise Animal or Animal-Derived (e.g. Mammal Milk) Matter.

As used herein the adjective “dairy” shall be taken to mean a composition or product comprises or consists of mammalian milk matter, i.e. the lacteal secretion obtainable by milking.

As used herein the terms “-free” or “free from” shall be taken to mean a composition or product which preferably does not contain a given substance but where trace amounts or contaminants thereof may be present.

As used herein the term “added sugar” shall refer to sugars that are added during the processing of foods (e.g. plant matter processed to provide a vegetal base) as opposed to sugars naturally occurring in said foods. Added sugars include sugars (free, mono- and disaccharides), sugars from syrups and honey, and sugars from concentrated fruit or vegetable juices that are in excess of what would be expected from the same volume of 100 percent fruit or vegetable juice of the same type.

As used herein, the term “fermented plant-based” shall be taken to mean a product or composition that is the product of the acidifying fermentation of a plant-based composition by a starter culture of fermenting microorganisms, in particular bacteria, preferably lactic acid bacteria.

As used herein, the term “fermented dairy milk” shall be taken to mean a product or composition derived from dairy milk by the acidifying action of at least one lactic acid bacterium, such as a yogurt (e.g., a set, stirred or drink yogurt), or a fresh cheese such as a white cheese or a “petit-Suisse”. It can be also be a strained fermented milk such as a strained yoghurt (e.g., a concentrated or Greek-style yoghurt).

As used herein the terms plant-based alternative, analogue or substitute shall be taken to mean a plant-based food or beverage composition that is formulated to simulate the organoleptic and/or nutritional qualities of an equivalent non plant-based product. Accordingly a “plant-based fermented milk alternative” shall be taken to mean a plant-based food or beverage composition that is formulated to simulate the organoleptic and/or nutritional qualities of fermented dairy milk. A “plant-based yogurt” shall be taken to mean a plant-based food or beverage composition that is formulated to simulate the organoleptic and/or nutritional qualities of fermented dairy yogurt.

The term “dairy yogurt” or “plant-based yogurt” as used herein shall be taken to mean fermented dairy or plant-based milk respectively obtained by the acidifying lactic fermentation of the bacteria Lactobacillus delbrueckii subsp. bulgaricus and Streptococcus thermophilus (also referred to as Streptococcus salivarius subsp. thermophilus), which must be viable in the finished product at a minimum CFU. In certain countries, regulations allow the addition of further lactic acid bacteria to yoghurt such as but not limited to strains of Bifidobacterium and/or Lactobacillus acidophilus and/or Lactobacillus casei. These additional lactic acid bacteria strains are intended to impart various properties to the finished product, such as that of providing organoleptic qualities, favoring equilibrium of intestinal flora or modulating the immune system.

The term “phytate degrading” as used herein shall be taken to mean the degradation of phytate sufficient to increase mineral bioavailability. Methods for the determination of phytate degradation are known in the art and typically include the detection of relevant minerals such as phosphorous. Commercially available assays suitable for such methods include the Megazyme K-PHYT kit.

Lactococcus lactis Strain

In a first aspect the present invention provides the strain Lactococcus lactis CNCM I-5450. This strain has been deposited at the Collection Nationale de Cultures de Microorganismes (CNCM) (Institut Pasteur, 25 Rue du Docteur Roux, Paris, France) under the Budapest Treaty on 20 Nov. 2019 under reference number CNCM I-5450. The deposit was made in accordance with the Budapest Treaty on the International Recognition of the Deposit of Microorganisms for the Purposes of Patent Procedure, as provided therein the applicant requests that a sample of the deposited micro-organisms only be made available to an independent expert, until the date on which the patent may be granted.

In one embodiment the present invention provides the isolated strain Lactococcus lactis CNCM I-5450, preferably said isolate is substantially pure.

### Compositions of the Invention

In a second aspect the present invention provides compositions comprising phytate degrading Lactococcus lactis, preferably CNCM I-5450.

Preferably, the composition comprises at least 10⁶, more preferably at least 10⁷ and most preferably at least 10⁸ colony forming unit (CFU) phytate degrading Lactococcus lactis, preferably CNCM I-5450, per gram (g) of composition according to embodiments of the invention.

In some embodiments, the composition comprises at least 10⁶, 10⁷, 10⁸, 10⁹, 10¹⁰, 10¹¹ or 10¹² colony forming unit (CFU) phytate degrading Lactococcus lactis, preferably CNCM I-5450, per gram (g) of composition according to embodiments of the invention.

In embodiments, the composition comprises 10⁵ to 10¹² colony forming unit (CFU) phytate degrading Lactococcus lactis, preferably CNCM I-5450 per gram (g) of composition according to embodiments of the invention. In further embodiments, the composition comprises 10⁶ to 10¹¹ colony forming unit (CFU) phytate degrading Lactococcus lactis, preferably CNCM I-5450 per gram (g) of composition according to embodiments of the invention.

The bacterium as provided herein is suitable for use in edible compositions, accordingly in one embodiment the present invention provides a composition suitable for human consumption or ingestion, preferably by oral means. Accordingly the composition comprises or consists of comestible matter. It is particularly preferred that the compositions of embodiments of the invention are substantially free of pathogenic or toxicogenic matter. The composition according to embodiments of the invention may be a medicament or pharmaceutical composition. In a particularly preferred embodiment the composition according to the invention may be a non-therapeutic composition, preferably a nutraceutical composition, a nutritional composition, nutritional supplement and/or a food composition. It is particularly preferred that the food composition is a fermented food composition, preferably a fermented dairy composition. Further compositions according to embodiments of the invention also include food additives, food ingredients, nutritional formulas, baby foods, infant milk formulas, infant follow-on formulas and young child formulas.

The composition may comprise further additional strains of Bifidobacterium and/or lactic acid bacteria; typically 2, 3, 4 or more additional strains. Examples of Bifidobacterium that can be used include but are not limited to Bifidobacterium animalis (for example Bifidobacterium animalis subsp. animalis or Bifidobacterium animalis subsp. lactis); Bifidobacterium longum; Bifidobacterium breve; Bifidobacterium bifidum. Examples of lactic acid bacteria that can be used include but are not limited to Lactobacilli (for example Lactobacillus acidophilus, Lactobacillus buchneri, Lactobacillus delbruckei, in particular L. delbrueckii subsp. bulgaricus or lactis, Lactobacillus casei, Lactobacillus plantarum, Lactobacillus reuteri, Lactobacillus johnsonii, Lactobacillus helveticus, Lactobacillus brevis, Lactobacillus rhamnosus); Lactococci (for example Lactococcus lactis, typically Lactococcus lactis subsp. lactis or Lactococcus lactis subsp. cremoris). Preferably the composition further comprises Lactobacillus and/or Streptococcus. For the preparation of yogurt, the composition typically comprises Lactobacillus bulgaricus (also referred to as Lactobacillus delbrueckii subsp. bulgaricus) and Lactococcus lactis, optionally with additional microorganisms such as but not limited to probiotic species or other species that may provide desirable organoleptic or other qualities to the composition, e.g. further strains of Lactococcus lactis.

Accordingly in one embodiment the present invention provides a composition comprising phytate degrading Lactococcus lactis, preferably CNCM I-5450, and further comprising at least one strain of Lactobacillus bulgaricus and optionally one or more strains of Lactococcus lactis and/or Bifidobacterium.

The strains of the present invention are particularly suited to the preparation of fermented compositions. Accordingly, in one embodiment the present invention provides a fermented food composition, preferably a fermented food product such as a fermented milk or plant-based product.

### Inoculum Compositions

The bacterium as described herein is useful as starter culture in the preparation of food compositions, such as fermented dairy products. Accordingly, in one embodiment the present invention provides an inoculum comprising Lactococcus lactis CNCM I-5450 that is suitable for the preparation of fermented dairy products. The inoculum of the invention is suitable for the direct inoculation Lactococcus lactis CNCM I-5450 into a composition comprising milk to provide fermented dairy products of the invention, typically without the need for a culture step prior to the said direct inoculation.

Typically the inoculum further comprises excipient or carriers, the selection of which is within the scope of the skilled person but may include buffers or culture media. The inoculum may optionally comprise further components such as cryoprotectants, preservatives and/or additives including nutrients such as yeast extracts, cysteine, sugars and vitamins.

Typically the inoculum is for use in the preparation of fermented dairy products, according in one embodiment the inoculum of the invention may be provided to the dairy composition in quantities of up to about 500 mg/I.

Typically the inoculum is fresh, frozen, dried or lyophilized. The inoculum may be in liquid, dry, spray-dried or solid form. It is particularly preferred that the inoculum is in liquid form. The inoculum may be defrosted and/or dispersed in liquid (e.g. water) prior to inoculation into a composition comprising milk.

In embodiments, the inoculum comprises at least 10⁹ cfu, e.g. at least 10¹⁰ cfu, such as at least 10¹¹ cfu Lactococcus lactis CNCM I-5450 per gram of inoculum composition. In embodiments, the inoculum comprises 10⁹ to 10¹² colony forming unit (CFU), or more preferably 10¹⁰ to 10¹² colony forming unit (CFU) Lactococcus lactis CNCM I-5450 per gram of inoculum.

Preferably the inoculum comprising Lactococcus lactis CNCM I-5450 is substantially pure.

In a further embodiment the present invention provides a mixture or kit of parts of the inoculum of the invention together with inoculum of Bifidobacterium and/or lactic acid bacteria.

Examples of Bifidobacterium that can be used include but are not limited to Bifidobacterium animalis (for example Bifidobacterium animalis subsp. animalis or Bifidobacterium animalis subsp. lactis); Bifidobacterium longum; Bifidobacterium breve; Bifidobacterium bifidum. Examples of lactic acid bacteria that can be used include but are not limited to Lactobacilli (for example Lactobacillus acidophilus, Lactobacillus buchneri, Lactobacillus delbrueckii, in particular L. delbrueckii subsp. bulgaricus or lactis, Lactobacillus casei, Lactobacillus plantarum, Lactobacillus reuteri, Lactobacillus johnsonii, Lactobacillus helveticus, Lactobacillus brevis, Lactobacillus rhamnosus); Lactococci (for example Lactococcus lactis, typically Lactococcus lactis subsp. lactis or Lactococcus lactis subsp. cremoris). Preferably the inoculum mixture further comprises Lactobacillus and/or Streptococcus. For the preparation of yogurt, the inoculum mixture typically comprises Lactobacillus bulgaricus (also referred to as Lactobacillus delbruckeii subsp. bulgaricus) and Lactococcus lactis, optionally with additional microorganisms such as but not limited to probiotic species or other species that may provide desirable organoleptic or other qualities to the composition, e.g. Lactococcus lactis.

Accordingly in one embodiment the present invention provides an inoculum mixture comprising a Lactococcus lactis CNCM I-5450 inoculum and further comprising at least one inoculum of Lactobacillus bulgaricus and optionally one or more additional inoculum of Lactococcus lactis and/or Bifidobacterium.

### Fermented Plant-Based Compositions

In a further aspect, the present invention provides fermented plant-based compositions comprising phytate degrading Lactococcus lactis, preferably CNCM I-5450.

In a first embodiment, the present invention provides compositions of the invention comprising i) a fermented vegetal base, ii) phytate degrading Lactococcus lactis, preferably CNCM I-5450.

In embodiments said composition does not comprise soy.

In embodiments the fermented compositions of the invention are free from, or do not comprise, raffinose, stachyose or verbacose.

In embodiments, the plant-based compositions of the invention comprise at least 10⁵ cfu/g, more preferably at least 10⁶ cfu/g, such as at least 10⁶ cfu/g, e.g. at least 10⁸ cfu/g, such as at least 10⁹ cfu/g, e.g. at least 10¹⁰ cfu/g, such as at least 10¹¹ cfu/g of ii) phytate degrading Lactococcus lactis, preferably CNCM I-5450

In embodiments the phytate degrading Lactococcus lactis, preferably CNCM I-5450 comprises or consists of CNCM I-5450.

The fermented plant-based compositions according to embodiments of the invention preferably comprise at least 10⁵, 10⁶, 10⁷, 10⁸ or 10⁹ CFU/g phytate degrading Lactococcus lactis, preferably CNCM I-5450. In embodiments, the plant-based compositions of the invention comprise 10⁵ to 10¹² or 10⁶ to 10¹⁰ colony forming unit (CFU) phytate degrading Lactococcus lactis, preferably CNCM I-5450 per gram of composition. In a most preferred embodiment the plant-based compositions comprise between 1×10⁶ and 2×10⁸ cfu/g phytate degrading Lactococcus lactis, preferably CNCM I-5450.

Preferably the fermented plant-based composition is prepared by culture of a vegetal base at a suitable temperature with phytate degrading Lactococcus lactis, preferably CNCM I-5450 to provide the required reduction in phytate, preferably by culturing for less than or equal to 12, 10, 8, 7, 6, 5 or 4 hours.

In embodiments the plant-matter comprises legumes, and most preferably, pulse or pulses. In embodiments the pulses are selected from the group consisting of split peas, field peas, dry peas, lentil, chickpeas, garbanzo bean, konda, navy bean, white navy bean, white pea bean, pea bean, cow pea, horse bean, haricot, pinot bean, mottled bean, small red bean, red Mexican bean, kidney bean, black bean, black turtle bean, cranberry bean, roman bean, speckled sugar bean, lima bean, haba bean, Madagascar bean, green gram, mung bean, green bean, black gram, urad dal, soy and/or lupin. In preferred embodiments, the pulses are pea and/or chickpea.

In embodiments the nuts are selected from the group consisting of almonds, cashews, pecans, macadamias, hazelnuts, pistachio, walnuts or combinations thereof.

In embodiments the seeds are selected from the group consisting of hemp, pumpkin, quinoa, sesame, tiger nut, flax, chia, sunflower, coconut or combinations thereof.

In embodiments said cereals are selected from the group consisting of wheat, rye, spelt, barley, oat, millet, sorghum, rice, teff and combinations thereof.

In preferred embodiments the plant-matter comprises chickpea, pat, buckwheat, lupin, millet, rice, coconut, soy and/or combinations thereof.

Particularly preferred is a base free from, or that does not comprise, added sugar, where the total carbohydrate content of the vegetal base is derived from plant-matter selected from the group consisting of phytate containing legumes, nuts, seeds, cereals and/or combination thereof.

In one embodiment the vegetal base is an aqueous suspension or slurry comprising water and plant-matter.

Processes for the preparation of such suspensions are known in the art and typically comprise mechanical and/or enzymatic disruption of the plant-matter and hydration and/or combination with a solution, followed by mechanical separation of an aqueous fraction from starchy and/or fibrous matter, e.g., by decentering, centrifugation or filtration.

For example, the plant-matter may be milled, ground, soaked, dehulled, mixed with water, optionally enzymatic hydrolysed and/or homogenized etc. in order to produce a suitable aqueous composition.

In embodiments the plant-matter has been subjected to a step of hydrolysis (e.g. enzymatic hydrolysis) and thus the vegetal base comprises fully or partially hydrolyzed hydrolysed plant-matter such as fully or partially hydrolyzed cereal.

In embodiments the plant matter may be a hydrolyzed cereal suspension such as an oat milk or syrup. Processes for the preparation of such cereal suspensions typically comprise mixing an oat material (such as rolled oats, milled oats, oat flour or oatmeal) with water and treated enzymatically by amylases to hydrolyze starch followed by removal of suspended matter.

In embodiments the plant matter may be a seed or nut butter such as sunflower, sesame, soy, almond, cashew, hazelnut or peanut butter. Processes for the preparation of nut butters typically comprise wet or dry grinding roasted or unroasted nuts to a paste having a particle size suitable for the preparation of nut beverages.

In particular embodiments, the vegetal base comprises a plant-based dairy analogue or dairy substitute beverage such as milk or cream preferably a plant-based milk, such as nut, oat or coconut milk.

Processes for the preparation of said beverages typically comprise the incorporation of suitable plant-based matter (e.g. oat syrup, nut butter) with water and other ingredients such as emulsifiers, stabilizing and flavoring agents. In particular embodiments, other ingredients may include one or more hydrocolloids (e.g., gellan gum, guar gum, locust bean gum, and xanthan gum), one or more salts (e.g., sea salt (e.g., sodium chloride), a potassium phosphate (e.g., monopotassium phosphate (KH₂PO₄), dipotassium phosphate (K₂HPO₄), tripotassium phosphate (K₃PO₄) etc.), a sodium phosphate (e.g., disodium phosphate (Na₂HPO₄)), a calcium phosphate (e.g., tricalcium phosphate Ca₃(PO₄)₂), and/or any other suitable emulsifying, flavoring, stabilizing, and/or buffering agent or combination of agents), and lecithin. Other ingredients may also include nutritional supplements such as vitamin A, vitamin B2, vitamin B12, vitamin D, vitamin E, zinc, fiber, protein, calcium, potassium, phosphorus, fatty acids, (e.g., omega 3, omega 6, etc.).

In one embodiment, it is preferred that the vegetal base does not contain animal, soy, gluten, dairy matter and/or combinations thereof.

In one embodiment, the vegetal base may be enriched or fortified with further components or nutrients such as but not limited to vitamins, minerals, trace elements or other micronutrients.

Preferably, the compositions of the invention comprise a protein content of at least about 2.5%, more preferably at least about 3% or 3.5%, most preferably 4%-5% (w/w).

Preferably, the composition has a pH equal to or lower than 5, 4.9, 4.8, 4.7 or most preferably equal to or lower than 4.6. In embodiments the composition has a pH preferably between about 4 and about 4.8, and more preferably between about 4.5 and about 4.8.

Preferably, the compositions of the invention has a viscosity lower than 200 mPa·s, more preferably lower than 100 mPa·s and most preferably lower that 60 mPa·s, at 10° C., at a shear rate of 64 s−1. In other embodiments, the composition has a viscosity range of 1 to 200 mPa·s, 1 to 100 mPa·s, or 1 to 60 mPa·s, at 10° C., at a shear rate of 64 s−1. In other embodiments, the composition has a viscosity range of 10 to 200 mPa·s, 10 to 100 mPa·s, or 10 to 60 mPa·s, at 10° C., at a shear rate of 64 s−1. In other embodiments, the composition has a viscosity range of 30 to 200 mPa·s, 30 to 100 mPa·s, or 30 to 60 mPa·s, at 10° C., at a shear rate of 64 s−1.

The fermented plant-based composition according to embodiments of the invention is preferably a food product, more preferably a plant-based fermented milk alternative. In embodiments said composition is an alternative of a product selected from the group comprising yogurt, set yogurt, stirred yogurt, pourable yogurt, yogurt drink, frozen yogurt, kefir, buttermilk, quark, sour cream, fresh cheese and cheese. In one embodiment, the composition is a drinkable composition, more preferably a plant-based alternative of a fermented milk drink such as but not limited to a yogurt drink, kefir etc. In an alternative embodiment, the composition is a composition that is spoonable, such as a plant-based alternative of a set or stirred yogurt or equivalent thereof.

In one embodiment, the fermented plant-based composition is a strained fermented plant-based composition.

Preferably, the fermented plant-based composition according to embodiments of the invention, may be stored, transported and/or distributed at a temperature of from 1° C. to 10° C. for at least about 30 days, at least about 60 days or at least about 90 days from packaging and remain suitable for consumption.

According to a further embodiment, the process for the preparation of a fermented product as defined above optionally comprises a stage of addition of an intermediate preparation as described above prior or subsequent to fermentation, said intermediate preparation typically comprising a preparation of fruits and/or cereals and/or additives such as flavorings and/or colourings.

### Fermented Milk Compositions

In a further aspect, the present invention provides fermented milk compositions comprising phytate degrading Lactococcus lactis, preferably CNCM I-5450.

In embodiments the fermented milk composition comprises at least about 30% (w/w) milk, more preferably at least about 50% (w/w) milk and even more preferably at least about 70% (w/w) milk. In embodiments, the composition comprises 30% to 100% (w/w) milk. In embodiments, the composition comprises 50% to 100% (w/w) milk. In embodiments, the composition comprises 70% to 100% (w/w) milk. Preferably said milk is vegetal and/or animal milk (dairy), more preferably soya, almond, oat, hemp, spelt, coconut, rice, goat, ewe, camel, mare or cow milk, and most preferably to cow milk.

Preferably said milk(s) are heat-treated, typically pasteurized, to ensure sterility. Preferably said heat treatment is carried out prior to the preparation of the fermented dairy composition.

Preferably said milk comprises one or more of skimmed, partially-skimmed or non-skimmed milk. Preferably said milk or milks may be in liquid, powdered and/or concentrated form. In one embodiment said milk further comprises milk components preferably selected from the group consisting of cream, casein, caseinate (for example calcium or sodium caseinate), whey proteins notably in the form of a concentrate (WPC), milk proteins notably in the form of a concentrate (MPC), milk protein hydrolysates, and mixtures thereof. In one embodiment said mixture further comprises plant and/or fruit juices. In one embodiment said milk or milks may be enriched or fortified with further milk components or other nutrients such as but not limited to vitamins, minerals, trace elements or other micronutrients.

In embodiments the dairy composition comprises above about 0.3 g per 100 g by weight free lactic acid, more preferably above about 0.7 g or 0.6 g per 100 g by weight free lactic acid. In embodiments, the composition comprises 0.3 g to 0.7 grams per 100 g by weight free lactic acid.

In embodiments the fermented milk composition comprises a protein content at least equivalent to that of dairy milk, preferably at least about 2.5%, more preferably at least about 3% or 3.5% (w/w). Preferably the composition has a pH equal to or lower than 5, preferably between about 3 and about 4.5 and more preferably between about 3.5 and about 4.5.

In embodiments the fermented milk composition has a viscosity lower than 200 mPa·s, more preferably lower than 100 mPa·s and most preferably lower that 60 mPa·s, at 10° C., at a shear rate of 64 s⁻¹. In embodiments, the composition has a viscosity range of 1 to 200 mPa·s, 1 to 100 mPa·s, or 1 to 60 mPa·s, at 10° C., at a shear rate of 64 s⁻¹. In embodiments, the composition has a viscosity range of 10 to 200 mPa·s, 10 to 100 mPa·s, or 10 to 60 mPa·s, at 10° C., at a shear rate of 64 s⁻¹. In embodiments, the composition has a viscosity range of 30 to 200 mPa·s, 30 to 100 mPa·s, or 30 to 60 mPa·s, at 10° C., at a shear rate of 64 s⁻¹.

In embodiments the fermented milk composition according to embodiments of the invention is preferably a product selected from the group comprising yogurt, set yogurt, stirred yogurt, pourable yogurt, yogurt drink, frozen yogurt, kefir, buttermilk, quark, sour cream, fresh cheese and cheese. In one embodiment the composition according to embodiments of the invention is a drinkable composition, more preferably a fermented milk drink such as but not limited to a yogurt drink, kefir etc. In an alternative embodiment the composition according to embodiments of the invention is a composition that is spoonable, such as a set or stirred yogurt or equivalent thereof.

In embodiments the fermented milk composition is a strained fermented dairy composition. The strained fermented dairy composition preferably has the following contents (% by weight):
from 8.5% to 11.0% of milk protein
from 0.0% to 8.0% of fat, for example from 0.0% to 3.5% or from 3.5% to 8.0%
from 0.00% to 4.20% of lactose, for example from 2.80% to 4.20%
   The pH of the strained fermented dairy composition can for example be of from 3.80 to 4.65.

Preferably the composition, according to embodiments of the invention, may be stored, transported and/or distributed at a temperature of from 1° C. to 10° C. for at least about 30 days, at least about 60 days or at least about 90 days from packaging and remain suitable for consumption.

In embodiments, the dairy compositions of the invention comprise at least 10⁵ cfu/g, more preferably at least 10⁶ cfu/g, such as at least 10⁷ cfu/g, e.g. at least 10⁸ cfu/g, such as at least 10⁹ cfu/g, e.g. at least 10¹⁰ cfu/g, such as at least 10¹¹ cfu/g Lactococcus lactis CNCM I-5450 per gram of dairy composition. In embodiments, the compositions of the invention comprise 10⁵ to 10¹² or 10⁶ to 10¹⁰ colony forming unit (CFU) Lactococcus lactis CNCM I-5450 per gram of composition.

Preferably, the composition is a packaged product that comprises at least 10⁶, more preferably at least 10⁷ and most preferably at least 10⁸ colony forming unit (CFU) Lactococcus lactis CNCM I-5450 per gram (g) of composition according to embodiments of the invention subsequent to storage, transport and/or distribution at a temperature of from 1° C. to 10° C. for at least about 30 days, at least about 60 days or at least about 90 days from packaging.

In embodiments, the composition is a packaged product that comprises 10⁵ to 10¹² or 10⁶ to 10¹⁰ colony forming unit (CFU) Lactococcus lactis CNCM I-5450 per gram (g) of composition according to embodiments of the invention subsequent to storage, transport and/or distribution at a temperature of from 1° C. to 10° C. for at least about 30 days, at least about 60 days or at least about 90 days from packaging.

In embodiments, the dairy composition further comprises an intermediate preparation. Intermediate preparations are known to the one skilled in the art. They are typically used to modify the taste, mouthfeel and/or texture of a dairy composition, for example of a fermented dairy composition. They can used also to introduce some additives such as nutrients. They typically comprise sweetening agents, flavors, color modifiers, cereals and/or fruit. Intermediate fruit preparations are for example slurries or fruit preparations. Flavors include for example fruit flavors, vanilla flavors, caramel flavors, coffee flavors, chocolate flavors.

Fruit preparations typically comprise fruits, as used herein the term “fruit” refers to any fruit form, including for example full fruits, pieces, purees, concentrates, juices etc.

The intermediate preparation or slurry typically comprises a stabilizing agent, having at least one stabilizer. The stabilizing agent can comprise at least two stabilizers. Such stabilizers are known to the one skilled in the art. They typically help in avoiding phase separation of solids, for examples of fruits or fruits extracts and/or in avoiding syneresis. They typically provide some viscosity to the composition, for example a viscosity (Bostwick viscosity at 20° C.) of from 1 to 20 cm/min, preferably of from 4 to 12 cm/min.

The stabilizing system or the stabilizer can for example be a starch, a pectin, an agar, a xanthan, a carrageenan, a locust bean gum, or a mixture thereof. The amount of stabilizing system is typically of from 0.5 to 5% by weight.

The intermediate preparation can typically comprise organoleptic modifiers. Such ingredients are known by the one skilled in the art.

The organoleptic modifiers can be for example sweetening agents different from sugar, coloring agents, cereals and/or cereal extracts.

Examples of sweetening agents are ingredients referred to as High Intensity Sweeteners, such as sucralose, acesulfamK, aspartam, saccharine.

Examples of fruits include for example strawberry, peach, apricot, mango, apple, pear, raspberry, blueberry, blackberry, passion, cherry, and mixtures or associations thereof, such as peach-passion.

The fruits can be for example provided as:

frozen fruit cubes, for example 10 mm fruit cubes, for example Individual Quick Frozen fruit cubes, for example strawberry, peach, apricot, mango, apple, pear fruit cubes or mixtures thereof,

Aseptic fruit cubes, for example 10 mm fruit cubes, for example strawberry, peach, apricot, mango, apple or pear fruit cubes or mixtures thereof,

fruit purees, for example fruit purees concentrated from 2 to 5 times, preferably 3 times, for example aseptic fruit purees, for example strawberry, peach, apricot, mango, raspberry, blueberry or apple fruit purees or mixtures thereof,

single aseptic fruit purees, for example strawberry, raspberry, peach, apricot, blueberry or apple single aseptic fruit purees or mixture thereof,

frozen whole fruits, for example Individual Quick Frozen whole fruits, for example blueberry, raspberry or blackberry frozen whole fruits, or mixtures thereof,

mixtures thereof.

The ingredients and/or components of the intermediate preparation and the amounts thereof can be typically such that the composition has a brix degree of from 1 to 65 brix, for example from 1 to 10 brix, or from 10 to 15 brix, or from 15 to 20 brix, or from 20 to 25 brix, or from 25 to 30 brix, or from 30 to 35 brix, or from 35 to 40 brix, or from 40 to 45 brix, or from 45 to 50 brix, or from 50 to 55 brix, or from 55 to 60 brix, or from 55 to 60 brix, or from 60 to 65 brix.

A fruit preparation can for example comprise fruit in an amount of from 30% to 80% by weight, for example from 50 to 70% by weight.

The intermediate preparation can comprise water. It is mentioned that a part of the water can come from ingredients used to prepare the fruit preparation, for example from fruits or fruit extracts or from a phosphoric acid solution.

The fruit preparation can comprise pH modification agents such as citric acid. The fruit preparation can have a pH of from 2.5 to 5, preferably of from 2.8 to 4.2.

Typically a fruit preparation can be added in an amount of 5-35% by weight with reference to the total amount of composition. In embodiments the composition of the invention comprises up to about 30% (w/w) of said intermediate preparation, e.g. up to about 10%, 15%, 20%, 25% (w/w). In one embodiment, the composition according to embodiments of the invention comprise 1% to 30% (w/w) of said intermediate preparation. In alternative embodiments, the composition according to embodiments of the invention comprise 1% to 25% (w/w) of said intermediate preparation. In further alternative embodiments, the composition according to embodiments of the invention comprise 1% to 20% (w/w) of said intermediate preparation. In additional embodiments, the composition according to embodiments of the invention comprise 1% to 15% (w/w) of said intermediate preparation. In further additional embodiments, the composition according to embodiments of the invention comprise 1% to 10% (w/w) of said intermediate preparation.

Preferably the composition, according to embodiments of the invention is provided in a sealed or sealable container containing about 50 g, 60 g, 70 g, 75 g, 80 g, 85 g, 90 g, 95 g, 100 g, 105 g, 110 g, 115 g, 120 g, 125 g, 130 g, 135 g, 140 g, 145 g, 150 g, 200 g, 300 g, 320 g or 500 g or about 1 oz, 2 oz, 3 oz, 4 oz, 5 oz, 6 oz or 12 oz product by weight.

In embodiments, the composition, according to embodiments of the invention is provided in a sealed or sealable container containing about 50 g to 500 g, 60 g to 500 g, 70 g to 500 g, 75 g to 500 g, 80 g to 500 g, 85 g to 500 g, 90 g to 500 g, 95 g to 500 g, 100 g to 500 g, 105 g to 500 g, 110 g to 500 g, 115 g to 500 g, 120 g to 500 g, 125 g to 500 g, 130 g to 500 g, 135 g to 500 g, 140 g to 500 g, 145 g to 500 g, 150 g to 500 g, 200 g to 500 g, 300 g to 500 g, 320 g to 500 g or 500 g product by weight. In embodiments, the composition, according to embodiments of the invention is provided in a sealed or sealable container containing about 1 oz to 12 oz, 2 oz to 12 oz, 3 oz to 12 oz, 4 oz to 12 oz, 5 oz to 12 oz, 6 oz to 12 oz or 12 oz product by weight.

Methods for the Preparation of Fermented Food Products

The bacteria as provided herein are suitable for use in the preparation of fermented food products. Accordingly an aspect the present invention also relates to the intended use of Lactococcus lactis, preferably CNCM I-5450, for the preparation of a food composition. In various embodiments the present invention provides a process for the preparation of food products comprising inoculating a food composition, such as a phytate containing food composition or a milk-base with Lactococcus lactis, preferably CNCM I-5450 and culturing.

In embodiments the present invention provides a process for the increase in mineral bioavailability in a phytate containing food comprising inoculating a phytate containing food composition with Lactococcus lactis, preferably CNCM I-5450 and culturing. In embodiments said culturing is carried out to provide a reduction of phytate of 0.5%, 5%, 10%, 15%, 20%, 25%, 30%, 35%, 40% w/w or more.

Accordingly in one embodiment the present invention provides a process for the reduction of phytate comprising the following steps:

a) providing a mixture comprising:

i) vegetal base comprising phytate

ii) phytate degrading Lactococcus lactis, preferably CNCM I-5450

b) culturing the mixture to provide a reduction of phytate.

In embodiments said culturing is carried out to provide a reduction of phytate of 0.5%, 5%, 10%, 15%, 20%, 25%, 30%, 35%, 40% w/w or more.

In embodiments the Lactococcus lactis produce phytase.

In embodiments the Lactococcus lactis preferably are capable of fermenting dairy milk to provide a reduction in pH to 5 or less, when inoculated at a rate of 10⁵ CFU/ml milk or higher when cultured at a temperature of 18° C. to 36° C. within 12 hours.

Vegetal bases as described above may be used in the processes of the invention. In preferred embodiments the plant-matter comprises chickpea, pat, buckwheat, lupin, millet, rice, coconut, soy and/or combinations thereof.

Preferably, fermented plant-based compositions are prepared using vegetal base that has been subjected to heat treatment at least equivalent to pasteurization. Preferably, the heat treatment is carried out prior to the preparation of the composition.

Fermentation of the mixture is carried out by incubating the mixture at a temperature suitable for the metabolization of the vegetal base by the bacteria to provide the desired reduction in phytate. Suitable temperatures for such fermentation are known to the person skilled in the art and for L. lactis strains may be between 18° C. to 36° C. The temperature is maintained for an incubation time sufficient to provide the desired reduction in phytate content.

Preferably the fermented plant-based composition is prepared by culture of the mixture to provide a reduction in pH, preferably to a pH equal to or lower than 5, 4.9, 4.8, 4.7 or 4.6. In embodiments the fermentation is carried out to a pH preferably between about 4 and about 4.8, and more preferably between about 4.5 and about 4.8. The pH can be adjusted by controlling the fermentation by the microorganism and stopping it when appropriate, for example by cooling.

However, as consumer acceptance of fermented plant-based food products can be challenging, in further embodiments the present invention provides a process for the preparation of a fermented dairy product comprising inoculating a milk-based composition with Lactococcus lactis CNCM I-5450 and fermenting.

Accordingly in one embodiment the present invention provides a process comprising the following steps:

a) providing a mixture comprising:

i) milk

ii) phytate degrading Lactococcus lactis, preferably CNCM I-5450

b) fermentation of said mixture to provide a fermented dairy product.

In embodiments said fermentation is carried out to provide a reduction of phytate of 0.5%, 5%, 10%, 15%, 20%, 25%, 30%, 35%, 40% w/w or more.

Preferably said a) milk comprises at least 10%, 20%, 30%, 40%, 50%, 60%, 70%, 80%, 90% or 100% of mammalian milk, most preferably cow, goat or sheep milk.

Preferably fermented dairy products are prepared using milk that has been subjected to heat treatment at least equivalent to pasteurization. Preferably said heat treatment is carried out prior to the preparation of the composition.

Typically, milk is pasteurized by means of the following successive steps:

1) standardization of fatty substances of the raw material so as to obtain a standardized substance,

2) enrichment with dried matter of the standardized substance obtained in the preceding stage, so as to obtain an enriched substance,

3) preheating of the enriched substance obtained in the preceding stage, so as to obtain a starting substance,

4) pasteurization and holding of the starting substance obtained in the preceding stage, so as to obtain a pasteurized and held substance,

5) an optional stage of homogenization of the pasteurized and held substance obtained in the preceding stage, so as to obtain a pasteurized, held and optionally homogenized substance,

6) initial cooling of the pasteurized, held and optionally homogenized substance obtained in the preceding stage, so as to obtain a pasteurized starting substance that has been held, optionally homogenized, and cooled down.

As used herein “standardization of fatty substances” is taken to mean a stage of bringing the quantity of fats present in the starting substance to a pre-determined level. Enrichment with dried matter involves the addition of proteins and fatty substance in order to modify curd firmness.

As used herein “holding” is taken to mean a rapid heating and maintenance of temperature of the milk and makes it possible to destroy the vegetative microbial flora, including pathogenic forms. Its typical duration is from 4 to 10 minutes, in particular from 5 to 8 minutes, and in particular approximately 6 minutes.

As used herein “homogenization” is taken to mean the dispersion of the fatty substances in the milk-type substance into small fat globules. The homogenization is carried out for example at a pressure of 100 to 280 bars, in particular 100 to 250 bars, in particular 100 to 200 bars, in particular approximately 200 bars. This homogenization stage is purely optional. It is in particular absent from the production process of products with 0% fatty substances.

Typically a fermented dairy product is prepared by culture of milks at a suitable temperature with suitable microorganisms to provide a reduction in pH, preferably to a pH equal to or lower than 5, preferably between about 3 and 4.7; more preferably between about 3.5 and about 4.7. The pH can be adjusted by controlling the fermentation by the microorganism and stopping it when appropriate, for example by cooling.

According to a further embodiment of the process for the preparation of a fermented dairy product as defined above, the mixture comprising milk and Lactococcus lactis CNCM I-5450 further comprises at least one, two, three or more strains of Bifidobacterium and/or lactic acid bacteria. The selection of suitable Bifidobacterium strains is within the scope of the skilled person and is typically a probiotic lactic acid bacteria. Examples of Bifidobacterium that can be used include but are not limited to Bifidobacterium animalis (for example Bifidobacterium animalis subsp. animalis or Bifidobacterium animalis subsp. lactis); Bifidobacterium longum; Bifidobacterium breve; Bifidobacterium bifidum.

The selection of suitable lactic acid bacteria strains is within the scope of the skilled person and is typically a thermophillic lactic acid bacteria. Examples of lactic acid bacteria that can be used include but are not limited to Lactobacilli (for example Lactobacillus acidophilus, Lactobacillus buchneri, Lactobacillus delbruckeii, in particular L. delbrueckii subsp. bulgaricus or lactis, Lactobacillus casei, Lactobacillus plantarum, Lactobacillus reuteri, Lactobacillus johnsonii, Lactobacillus helveticus, Lactobacillus brevis, Lactobacillus rhamnosus); Lactococci (for example Lactococcus lactis, typically Lactococcus lactis subsp. lactis or Lactococcus lactis subsp. cremoris). Typically a mixture or association of a plurality of species of lactic acid bacteria may be used, typically a mixture or association of Lactobacillus and Streptococcus. For the preparation of yogurt this typically includes Lactobacillus bulgaricus (also referred to as Lactobacillus delbrueckii subsp. bulgaricus) and Lactococcus lactis, optionally with additional microorganisms such as but not limited to probiotic species or other species that may provide desirable organoleptic or other qualities to the composition, e.g. Lactococcus lactis.

Accordingly in one embodiment the mixture further comprises at least one strain of Lactobacillus bulgaricus and optionally one or more strains of Lactococcus lactis and/or Bifidobacterium.

Suitable temperatures for milk fermentation are typically about 36° C. to about 44° C. and the temperature is maintained for an incubation time sufficient to provide the desired reduction in pH. For the preparation of a fermented dairy product the temperature at the start of fermentation is typically about 36° C. to about 43° C., in particular about 37° C. to about 40° C., the temperature at the end of fermentation is typically about 37° C. to about 44° C., in particular about 38° C. to about 41° C. The fermentation time is typically about 6 to about 11 hours.

Subsequent to the fermentation the fermented milk is cooled. Optionally a stage of intermediate cooling of the fermented milk may be performed to provide a pre-cooled fermented milk having a temperature of between about 22° C. and about 4° C. Typically the intermediate cooling time is about 1 hour to about 4 hours, in particular about 1 hour 30 minutes to about 2 hours. The pre-cooled fermented milk is typically stored for up to 40 hours or less.

Preferably a stage of final cooling of the fermented milk is performed such that the temperature at the start of the final cooling is less than about 22° C. and the temperature at the end of the final cooling is about 4° C. to about 10° C. The cooled product may then be stored, transported and/or distributed at a temperature from about 1° C. to about 10° C. for at least about 30 days, at least about 60 days or at least about 90 days.

According to a further embodiment, the process for the preparation of a fermented dairy product as defined above optionally comprises a stage of stirring at a pressure of at least 20 bars, or performing a dynamic smoothing, to obtain a composition having the desired viscosity, typically a viscosity of up to 20 mPa·s. Stirring or dynamic smoothing operations provide some shear to composition that typically allow a viscosity drop. Such operations are known by the one skilled in the art, and can be operated with conventional appropriate equipment. This stage is typically performed at cold temperature, for example at a temperature of form 1° C. to 20° C. Without intending to be bound to any theory, it is believed that applying some shear at cold temperature, typically by stirring at high pressure or by performing a dynamic smoothing, can lead to a fluid gel formation within the composition, that provides improved stability even at a low viscosity of up to 20 mPa·s.

Alternatively, according to a further embodiment, the process for the preparation of a fermented dairy product as defined above optionally comprises a stage of acid whey removal to provide a “strained fermented dairy composition”. In this step an acid whey composition is separated from the curd resulting from the protein coagulation due to acidification during fermentation. Thus one obtains:
a fermented dairy product, typically comprising the proteins coagulum, referred to as a strained fermented dairy composition, and
an acid whey by-product

Such separation steps are known by the one skilled in art, for example in processes of making “greek yogurts”. The separation can for example be carried out by reverse osmosis, ultrafiltration, or centrifugal separation. The separation step can be performed for example at a temperature of from 30° C. to 45° C.

According to a further embodiment, the process for the preparation of a fermented dairy product as defined above optionally comprises a stage of addition of an intermediate preparation as described above prior or subsequent to fermentation, said intermediate preparation typically comprising a preparation of fruits and/or cereals and/or additives such as flavorings and/or colourings.

Uses of the Invention

In an aspect, the present invention provides the use of Lactococcus lactis, preferably CNCM I-5450, for increasing bioavailability of phytate bound minerals of a food product.

The capacity of Lactococcus lactis to increase bioavailability of phytate bound minerals can be determined by techniques known in the art, such as those described in the examples. In an embodiment, the Lactococcus lactis according to the invention is added to the food product before its consumption by an individual.

In an embodiment, the Lactococcus lactis is administered to a healthy individual.

In another aspect the present invention provides Lactococcus lactis, preferably CNCM I-5450, for use for preventing or treating at least one symptom of mineral deficiency or insufficiency in an individual.

In embodiments, the Lactococcus lactis according to the invention is used for preventing or treating at least one symptom of mineral deficiency or insufficiency. In embodiments, the Lactococcus lactis according to the invention is used increasing bioavailability of phytate bound minerals.

In embodiments, the mineral is selected from the group consisting of calcium, iron, copper, magnesium, manganese or zinc. In embodiments the mineral-malnutrition is iron-deficiency anemia, calcium deficiency or zinc deficiency.

The present invention also relates to a method for preventing or treating at least one symptom of mineral deficiency or insufficiency in an individual in need thereof, comprising administering an effective amount of a composition comprising Lactococcus lactis to the individual.

The present invention also relates to the use of Lactococcus lactis in the manufacture of a medicament for preventing or treating at least one symptom of mineral deficiency or insufficiency.

The present invention also relates to a method for preventing or treating at least one symptom of mineral deficiency or insufficiency and/or increasing bioavailability of phytate bound minerals, comprising administering an effective amount of a composition comprising Lactococcus lactis to the individual prior to, concurrently with or subsequent to the consumption of at least one phytate containing food.

The “individual” according to the invention is preferably a mammal. In particular, it can be a farm animal such as a bovine, an ovine or a caprine, or a pet, such as a dog, a cat, a rabbit or a rodent. Most preferably, the individual according to the invention is a human.

In an embodiment, the individual according to the invention may suffer from at least one symptom of mineral deficiency or insufficiency.

In preferred but non limitative embodiments, said at least one symptom of mineral deficiency or insufficiency is selected from the group comprising: tingling, muscle cramps, seizures, numbness, poor appetite, loss of appetite, irregular heart rhythms, nausea, vomiting, tiredness or weakness.

In a particular embodiment of the invention, the individual is considered as being part of the general population or healthy.

Preferably, the individual according to the invention follows a plant-based diet.

Preferably, the composition according to the invention is used for preventing or treating at least one symptom of mineral deficiency or insufficiency consecutive to consumption of phytate containing food. In one embodiment said phytate containing food is comprised in the composition according to the invention. In an alternative embodiment the present invention provides a method for preventing or treating at least one symptom of mineral deficiency or insufficiency prior to or subsequent to the consumption of at least one phytate containing food in an individual, comprising administering an effective amount of a composition comprising Lactococcus lactis to the individual prior to or subsequent to the consumption of at least one phytate containing food. In a further alternative embodiment the present invention provides the use of a composition comprising Lactococcus lactis for preventing or treating at least one symptom of mineral deficiency or insufficiency prior to or subsequent to the consumption of at least one phytate containing food in an individual.

Preferably, the phytate containing food according to the invention comprises or consists of at least one fermentable ingredient, such as a carbohydrate.

In one embodiment the present invention provides the consumption or administration of a dose of between about 10⁸ and about 10¹¹ colony forming unit (CFU) of Lactococcus lactis, preferably between about 10⁸ and about 10⁹, more preferably between about 10⁹ and about 10¹⁰ colony forming unit (CFU) and in an alternative embodiment between about 10¹⁰ and about 10¹¹ colony forming unit (CFU) of Lactococcus lactis, preferably CNCM I-5450. In a further embodiment at least 1, 2, 3, or 4 doses are provided within a 24 hour time period. It is further preferred that the daily dosage regimen is maintained for at least about 1, 2, 3, 4, 5, 6 or 7 days, or in alternative embodiment for at least about 1, 2, 3, 4, 5, 6 or 7 weeks.

Accordingly, in one embodiment the present invention provides the daily consumption or administration of at least 1, 2, 3, or 4 servings of the compositions of the invention, in particular the fermented dairy composition according to the invention or the product according to the invention. Each serving may be consumed or administered individually, or a plurality of servings may be consumed or administered in a single instance. Each of said servings may be consumed at mealtimes or between mealtimes (e.g. as a snack, subsequent to sporting activities etc. . . . ).

A single serving portion of the dairy composition, in embodiments the fermented dairy composition according to the invention is preferably about 50 g, 60 g, 70 g, 75 g, 80 g, 85 g, 90 g, 95 g, 100 g, 105 g, 110 g, 115 g, 120 g, 125 g, 130 g, 135 g, 140 g, 145 g, 150 g, 200 g, 300 g or 320 g or about 1 oz, 2 oz, 3 oz, 4 oz, 5 oz, 6 oz or 12 oz by weight.

Preferably, the composition according to the invention comprises at least 10⁶, more preferably at least 10⁷ and most preferably at least 10⁸ colony forming unit (CFU) of Lactococcus lactis, preferably CNCM I-5450, according to the invention per gram (g) of composition according to the invention. Preferably also, the composition according to the invention comprises at least 10¹¹, more preferably at least 10¹⁰ and most preferably at least 10⁹ colony forming unit (CFU) of Lactococcus lactis, preferably CNCM I-5450, bacteria per gram (g) of composition according to the invention.

In embodiments the Lactococcus lactis reduce phytate and preferably are capable of fermenting dairy milk to provide a reduction in pH to 5 or less, when inoculated at a rate of 10⁵ CFU/ml milk or higher when cultured at a temperature of 18° C. to 36° C. within 12 hours.

The invention will be further illustrated by the following non-limiting Figures and Examples.

### DESCRIPTION OF THE FIGURES

FIG. 1 provides the phytate content of cereal flours determined according to Example 2.

FIG. 2 provides the % dephosporylation activity of bacteria CNCM I-5450 in cereal flours according to according to Example 2.

FIG. 3 provides the milk acidification kinetics of bacterial strains tested according to Example 3.

FIG. 4 provides the milk acidification kinetics of CNCM I-5450 tested according to Example 3.

FIG. 5 provides the population of bacteria CNCM I-5450 determined according to Example 4

FIG. 6 provides the % dephosporylation activity of bacteria CNCM I-5450 during the gastrointestinal tests according to Example 4.

### EXAMPLES

### Example 1: Screening of Phytate-Degrading Bacterial Strains

Approximately 900 individual bacterial strains from the Applicant's Danone Culture Collection were screened for phytates degradation activity of which 66 strains were identified as having phytates-degrading activities.

### Materials & Methods:

Bacterial strains were growth overnight at 37° C. in 96-wells microplate in a defined medium without phosphate source. A neutral MRS medium was modified to reduce its phosphate concentration. This modification was intended to allow the Inventors to measure the phosphate released by phytate degradation without reaching the saturation threshold of the standard range. To achieve this they removed fractions containing high concentrations of phosphate such as yeast extract and potassium phosphate. To compensate for these elements, a mixture of vitamins B and iron sulfate was added. After fermentation, cultures or supernatants were incubated in presence of sodium phytate and the total available phosphorus released from samples was measured. A quantitative method to measure total “available phosphorus” released from samples was used. Phytase activity was measured in terms of inorganic phosphate released from phytic acid by strains using a Megazyme kit assay.

Only the strains that showed the highest activity in the first screening (66) were chosen for subsequent experiments done in triplicate.

The inventors used kit from Megazyme (K-PHYT). This method used a quantification of phosphorous with a colorimetric assay. The amount of molybdenum blue formed in this reaction is proportional to the amount of inorganic phosphate (Pi) present in the sample and is measured by the increase in absorbance at 650 nm. Pi is quantified as phosphorus from a calibration curve generated using standards of known phosphorus concentration.

A reaction mixture containing 100 μl of cell suspension and 50 μl of phytate substrate (3 mM in acetate buffer) was incubated for 1 h at 37° C. Then the release of inorganic phosphate was measured by adding color reagent, prepared daily, and an incubation 1 h at 37° C. before reading the absorbance at 650 nm.

Results were compared to a standard curve prepared with inorganic phosphate (K2HPO4).

TABLE 1
Screening Results

| | Total number of | Phytate -degrading |
|---|---|---|
| Genus/Species | strains tested | strains |
| L. plantarum | 246 | 23 |
| L. rhamnosus | 67 | 2 |
| L. lactis | 142 | 5 |
| L. fermentum | 25 | 1 |
| Leuconostoc | 23 | 8 |
| Bifidobacterium | 254 | 11 |
| Pediococcus | 60 | 1 |
| L. brevis | 16 | 12 |
| L. reuteri | 5 | 0 |
| L. amylovorus | 4 | 0 |
| L. curvatus | 24 | 3 |
| L. sakei | 1 | 0 |
| | 867 | 66 |

### Example 2: Preparation of Fermented Vegetal Product

The 10 most effective phytate degrading strains from Table 1 were tested for their suitability for the degradation of phytate in plant-based food products as determined by phytate dephosphorylation activity in various plant flours.

Materials & Methods:

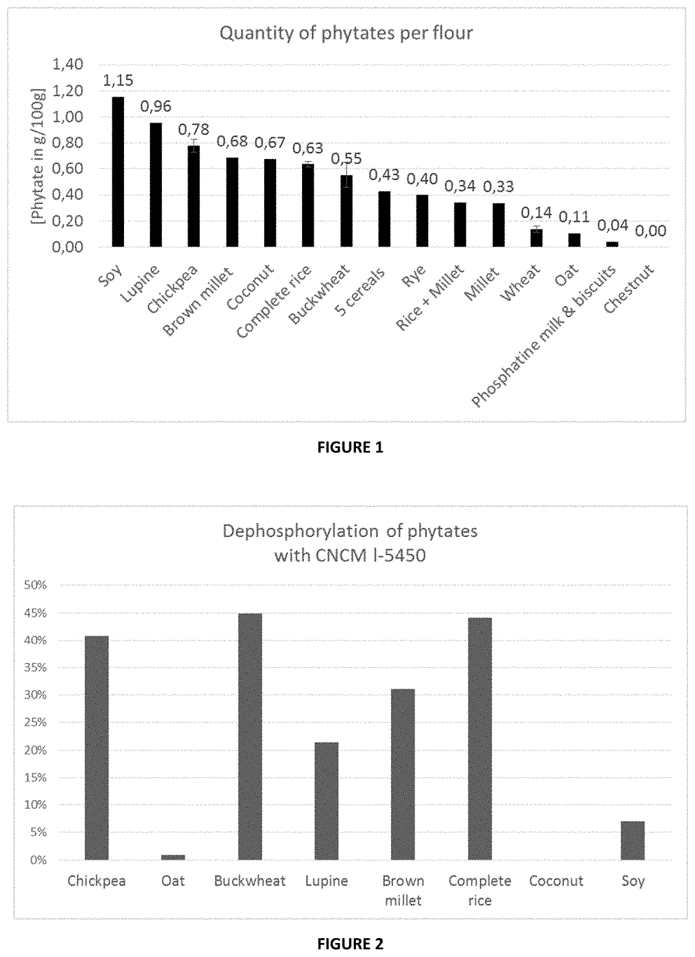

15 flours were evaluated for the amount of phytate, and it was decided to test the highest phytate containing flours: soy, lupine, chickpea, brown millet, coconut, complete rice, buckwheat and oat (see FIG. 1).

Flours were sterilized under UV light. For vegetal fermentation, 15 g of flour was suspended in 100 ml of distilled water. Fermentation was started by inoculation with 1% of overnight culture of individual lactic acid bacteria or bifidobacteria. Fermentation was done for 24 h at 37° C. Enzyme activities was measured at 37° C.

Prior to fermentation with strains, phytate contained in the different flours was determined by suspending 1 g of flour in 20 mL of hydrochloric acid (0.66 M) and stirred overnight at room temperature. The solution was neutralized by adding 0.75 ml of sodium hydroxyde. Inorganic phosphate total and released allowed the calculation of the amount of phytate in the sample. Selected strains were incubated overnight at 37° C. on each flour in 96-wells plate before testing the amount of phosphorous released during fermentation vs control without bacteria. During culture strain growth was observed using pH as an indicator as the tested strains produce acid that decreases the pH of the media. A further test of growth is the determination of the amount of strain after fermentation i.e. the population determined as cfu (colony forming unit) per ml. The bacteria in the fermented flours were enumerated using MRS agar medium, supplemented with cysteine (0.3 g/l). Plates were incubated under anaerobic conditions at 37° C. for 24 h.

After fermentation, quantification of phosphorous released during fermentation from phytate was measured. The amount of phytate in each selected flour was determined by mixing 1 g of flour with 20 mL of hydrochloric acid (0.66 M) and stirred overnight at room temperature.

Results:

Table 2 provides the % dephosphorylation of the best-performing strains in key flours, where no observable growth/fermentation was observed in a flour fields are left blank.

FIG. 2 provides the phytate dephosphorylation of key tested flours using strain CNCM I-5450.

TABLE 2

| | | | | | Brown | Unrefined | | |
|---|---|---|---|---|---|---|---|---|
| | Chickpea | Oat | Buckwheat | Lupin | Millet | rice | Coconut | Soy |
| Bifidobacteria | | | 52% | | 2% | | 62% | |
| 1 | | | | | | | | |
| Leuconostoc | 29% | 7% | 46% | | 32% | | 37% | 10% |
| Bifidobacteria | 11% | 12% | 40% | | | | 56% | 64% |
| 2 | | | | | | | | |
| Bifidobacteria | 38% | 5% | 26% | | 46% | | 48% | |
| 3 | | | | | | | | |
| Lactobacillus | | 9% | 39% | | 17% | 41% | 51% | 14% |
| CNCM I-5450 | 41% | 1% | 46% | 21% | 31% | 44% | | 7% |

Most strains grew well in all flours, surprisingly it was observed that the ability to dephosphorylate phytate was strain dependent.

### Example 3: Preparation of Fermented Dairy Milk Product

The 10 most effective phytate degrading strains from Table 1 were also tested for their suitability for the preparation of dairy fermented milk products.

Materials & Methods:

Fermented milk test products were prepared by preparing a milk base (135 g/L powdered milk, 0.2% yeast extract, 5% galactose, 0.03% cysteine) with 1% vol/vol bacterial culture (about 10⁶ CFU/ml). Fermentation was carried out at 37° C. and monitored using a CiNAC probe.

The aim was to identify phytate degrading strains, ideally combining the capacity to reduce phytate in relevant plant foodstuffs and also able to grow in milk. The aim was to identify strains that were effective in acidifying dairy milk, that could be used to prepare fermented dairy milk products (typically pH lower than 5).

Results:

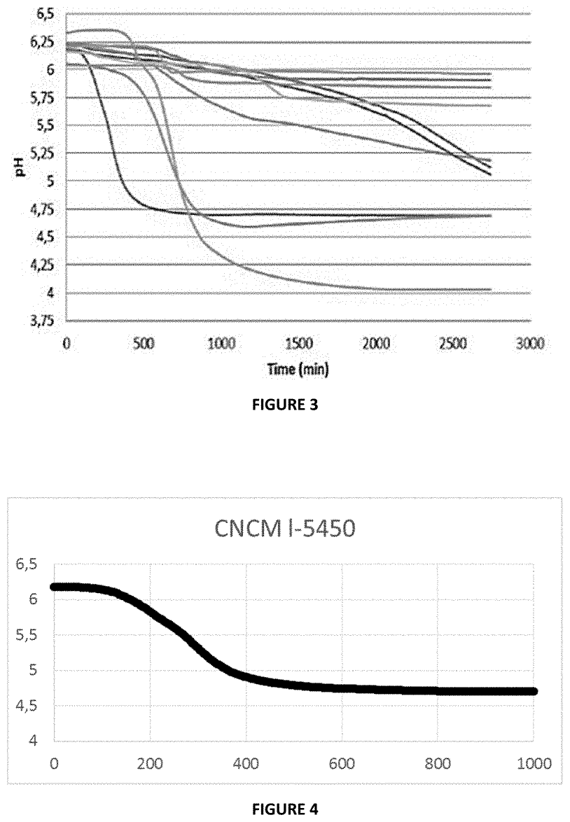

Only three of the strains were able to reduce the starting pH of the milk base (above 6) to lower than 4.75 within the target of 24 hours (extended fermentation times increases contamination risk), said strains achieving the target reduction within about 17 hours (see FIG. 3). Milk acidification kinetics of CNCM I-5450 are provided in FIG. 4.

### Example 4: In Vitro Model of Strain Survival

The inventors aimed to identify strains that not only had a phytate-degrading enzymatic activity, but also good survival capability in the digestive tract in order to provide probiotic strains. For that reason, strains survival in the gastric & intestinal systems was determined using in vitro models.

These models were also used to confirm if the phytate dephosphorylation activity was present in said gastrointestinal models.

The aim was to evaluate the tolerance of strains toward stomach acidic pH, bile salts by using an in vitro static test.

Fermented milks were incubated with or without phytate in gastric & intestinal fluid models to verify the cell survival and to estimate the capacity of strains to resist to gastric and intestinal conditions. During incubation, sodium phytate was added to the reaction mixture to assay phytase activity during digestion. A strain numeration was done at time points (see Figures) to measure the survival of the strain. MRS-cys broth was used and incubated at 37° C. for 24 h.

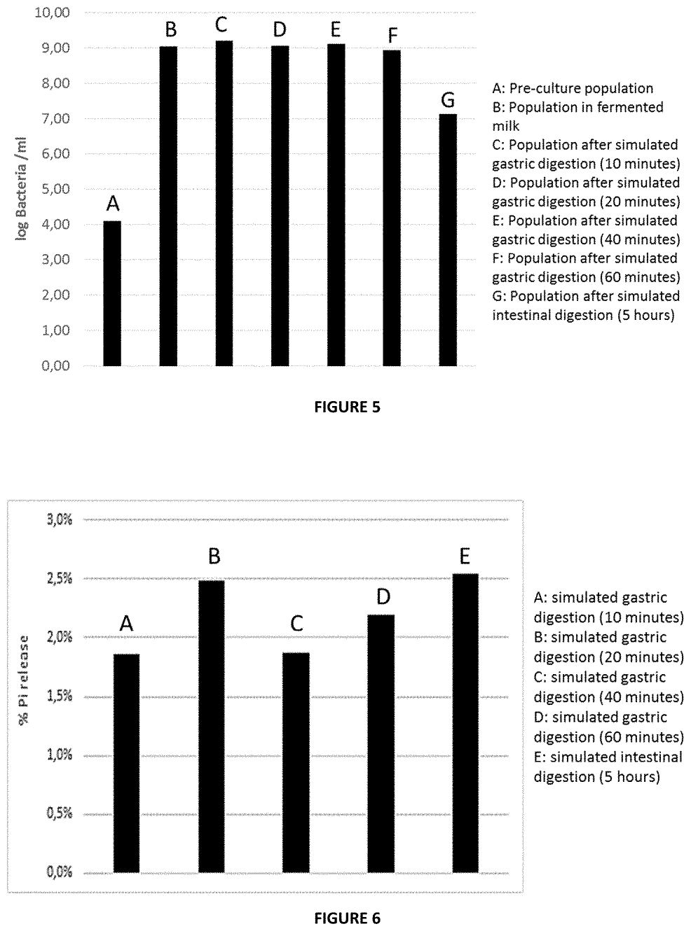

FIG. 5 provides the population of bacteria prior to (pre-culture & in fermented milk) and during the in vitro gastrointestinal tests (at various timepoints).

FIG. 6 provides the % dephosporylation activity during the gastrointestinal tests.

The strain shows a tolerance to gastric and intestinal conditions with a minimal decrease in cfu during intestinal stress and maintains some dephosphorylation capacity.

1. A method for preventing or treating at least one symptom of mineral deficiency or insufficiency in a subject, comprising administering an effective amount of a composition comprising L. lactis to the subject.

2. The method of claim 1, wherein said mineral is calcium, iron, copper, magnesium, manganese or zinc.

3. The method of claim 1, wherein said subject follows a plant-based diet.

4. The method of claim 1, wherein said L. lactis increase bioavailability of phytate bound minerals.

5. The method of claim 1, wherein said L. lactis are capable of fermenting a dairy milk.

6. The method of claim 1, wherein said L. lactis is CNCM I-5450.

7. L. lactis strain deposited at the CNCM under reference number CNCM I-5450.

8. A composition comprising at least 10⁵ CFU/g of the L. lactis strain of claim 7.

9. The composition according to claim 8, wherein said composition is a fermented composition.

10. The composition according to claim 8, wherein said composition comprises vegetal and/or dairy milk.

11. A method for increasing bioavailability of phytate bound minerals of a food product, comprising adding the L. lactis strain of claim 7 to the food product.

12. A process for the reduction of phytate, comprising:
culturing a mixture comprising a vegetal base comprising phytate, and a phytate degrading L. lactis, to provide a reduction of phytates.

13. The process of claim 12, wherein said culturing is carried out to provide a reduction in pH to 4.7 or lower.

14. The process of claim 12, wherein the L. lactis is CNCM I-5450.
